(19) United States
(12) Patent Application Publication
(10) Pub. No.: US 2026/0272273 A1
(43) Pub. Date: Sep. 17, 2026

(54) ARTICULATING MEDICAL DEVICES AND ASSOCIATED COMPONENTS, ASSEMBLIES, AND METHODS

(71) Applicant: Boston Scientific Scimed Inc., Maple Grove, MN (US)

(72) Inventors: Daniel Joseph GRASSO, Franklin, MA (US); Konstantin KOPOTIYENKO, Dedham, MA (US)

(73) Assignee: Boston Scientific Scimed Inc., Maple Grove, MN (US)

(21) Appl. No.: 19/561,698

(22) Filed: Mar. 10, 2026

Related U.S. Application Data
(60) Provisional application No. 63/771,366, filed on Mar. 13, 2025.

(51) Int. Cl. A61B 1/005; A61B 1/00

(52) U.S. Cl. CPC A61B 1/0055; A61B 1/00073; A61B 1/0008

(57) ABSTRACT

A medical device including a shaft defining at least one lumen, an articulation member extending through the at least one lumen, and a distal tip coupled to a distal end of the shaft. The distal tip includes a distal cap and a steering ring. The distal cap may have a distal portion and a proximal portion. The proximal portion includes a proximal extension. The steering ring at least partially surrounds the proximal extending of o the distal cap. The proximal extension of the distal cap defines a plurality of grooves. Each groove of the plurality of grooves is configured to receive a distal end of the articulation member. The plurality of grooves is defined along a radially outer surface of the proximal extension.

### CROSS-REFERENCE TO RELATED APPLICATIONS

This application claims the benefit of priority from U.S. Provisional Application No. 63/771,366, filed on March 13, 2025, which is incorporated by reference herein in its entirety.

### TECHNICAL FIELD

This disclosure relates generally to assemblies for articulating medical devices and associated components, assemblies, and methods. More specifically, aspects of this disclosure pertain to assemblies including components that are positioned at a distal tip of an articulating medical device, such as an endoscope.

### BACKGROUND

In a medical procedure, an operator may insert a medical device, such as an endoscope or other type of scope, into a body lumen of a subject. The operator may navigate the distal tip to a location where the procedure is to be performed. The operator may pass accessory devices (e.g., instruments) through a working channel of the medical device to assist with performing certain diagnostic or therapeutic procedures. The distal tip of the medical device may include elements for providing visualization of the body lumen, such as lighting elements and/or imaging devices. The distal tip of the medical device also may include other features, such as one or more articulation members, an opening of the working channel, one or more openings for air/water and suction, a sensor, and/or an elevator. Many current scopes present limitations on the arrangement of elements and/or types of elements that may be incorporated into the distal tip of the device. It may be desirable to arrange the articulation members within the distal tip to optimize the space within the distal tip and without increasing an outer diameter of the medical device. For example, it may be desirable to provide room for additional components and/or larger components (e.g., additional and/or larger lighting elements, imaging devices, and/or sensors).

### SUMMARY

Each of the aspects disclosed herein may include one or more of the features described in connection with any of the other disclosed aspects.

Aspects of this disclosure relate to, among other things, assemblies for medical devices and associated methods. Aspects of this disclosure pertain to assemblies including components that are positioned at a distal tip of a medical device, such as an endoscope.

According to an example, a medical device may include a shaft defining at least one lumen, an articulation member extending through the at least one lumen, and a distal tip coupled to a distal end of the shaft. The distal tip may include a distal cap and a steering ring. The distal cap may have a distal portion and a proximal portion. The proximal portion may include a proximal extension. The steering ring may at least partially surround the proximal extension of the distal cap. The proximal extension of the distal cap may define a plurality of grooves each configured to receive a distal end of the articulation member. The plurality of grooves may be defined along a radially outer surface of the proximal extension.

The medical devices discussed above and elsewhere may include any of the following features. The plurality of grooves may include a first groove and a second groove. The distal tip may further include a first cutout and a second cutout. The first cutout may be proximal of the first groove. The second cutout may be proximal of the second groove. Each of the first cutout and the second cutout may extend radially inward from an outer surface of the proximal extension through an inward-facing surface of the proximal extension. The articulation member may extend radially inward and through the first cutout and the second cutout.

Each of the first cutout and the second cutout may be defined by two parallel surfaces and a distal surface. The first cutout and the second cutout may be U-shaped when viewed from a side of the distal cap. The plurality of grooves may include a third groove and a fourth groove. The third groove and the fourth groove may be mirror images of one another relative to a central plane of the distal cap.

A distal end of each of the first groove, the second groove, the third groove, and the fourth groove may be defined by a proximal-facing wall. A longitudinal length of each of the third groove and the fourth groove may be greater than a longitudinal length of each of the first groove and the second groove. Each of the third groove and the fourth groove may be closer to a central longitudinal axis of the distal tip than the first groove and the second groove.

Each of the first groove and the second groove may be offset from respective lumens of the shaft. The third groove and the fourth groove may be aligned with respective lumens of the shaft.

The medical device may further include a spacer disposed proximal of the distal cap. The spacer may at least partially surround a distal portion of the shaft. The steering ring may at least partially surround a distal end portion of the spacer. A proximal portion of the spacer may taper radially inward proximally.

The distal cap may further define a first opening and at least one second opening. The first opening may be configured to receive an imaging device. The at least one second opening may be configured to receive an illumination device. The first opening may be separated from the second opening by a wall. Proximal of the wall, the first opening and the second opening may converge.

The distal end of the articulation member may be coupled to an internal surface of the steering ring.

According to another example, a distal end of a medical device may include a distal cap and a steering ring. The distal cap may have a distal portion and a proximal portion. The proximal portion may include a proximal extension. The proximal extension of the distal cap may define a groove defined along a radially outer surface of the proximal extension and a cutout proximal of the groove. The groove may be configured to receive a distal end of an articulation member. A portion of the articulation member may extend radially inward and through the cutout. The steering ring may at least partially surround the proximal extension of the distal cap.

The distal ends of medical devices discussed above and elsewhere may include any of the following features. The groove may be a first groove and the cutout may be a first cutout. The distal cap may further include a second groove, a third groove, and a fourth groove. The first groove and the second groove may be mirror images of one another relative to a central plane of the distal cap. The third groove and the fourth groove may be mirror images of one another relative to a central plane of the distal cap.

The distal end of the medical device may further include a spacer disposed proximal of the distal cap. The spacer may be configured to at least partially surround a distal portion of a shaft. The steering ring may at least partially surround a distal end portion of the spacer.

According to another example, a distal cap of a medical device may include a distal portion and a proximal portion and a steering ring. The proximal portion may include a proximal extension. The proximal extension of the distal cap may include a groove and a cutout proximal of the groove. The groove may be defined along a radially outer surface of the proximal extension. The groove may be configured to receive a distal end of an articulation member. A portion of the articulation member may be configured to extend radially inward and through the cutout. The steering ring may at least partially surround the proximal extension of the distal cap. The steering ring may be configured to couple the distal cap to a shaft of the medical device.

The distal cap of medical devices discussed above and elsewhere may include the following feature. The distal cap may further include a spacer disposed proximal of the distal cap. The spacer may be configured to at least partially surround a distal portion of a shaft. The steering ring may at least partially surround a distal end portion of the spacer.

The foregoing general description of the illustrative embodiments and the following detailed description thereof are merely exemplary aspects of the teachings of this disclosure and are not restrictive.

### BRIEF DESCRIPTION OF FIGURES

The accompanying drawings, which are incorporated in and constitute a part of this application, illustrate examples of this disclosure and together with the description, serve to explain the principles of the disclosure.

FIGS. 1A and 1B illustrate proximal and distal portions, respectively, of an exemplary medical device, according to aspects of this disclosure, according to aspects of this disclosure.

FIG. 2 illustrates a cross-sectional view of the distal portion of the medical device shown in FIG. 1B, according to aspects of this disclosure.

FIGS. 3A illustrates a proximal-to-distal view of a portion of the distal portion of the medical device shown in FIGS. 1B and 2, and FIG. 3B illustrates a perspective view of a portion of the distal portion of the medical device shown in FIGS. 1B and 2, according to aspects of this disclosure.

FIG. 4 illustrates a perspective view of a spacer of the distal portion of the medical device shown in FIGS. 1B and 2, according to aspects of the disclosure.

FIG. 5 illustrates a cross-sectional view of the distal portion of the medical device shown in FIGS. 1B and 2, according to aspects of this disclosure.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The drawings may include arrows labeled “P” and “D,” indicating proximal and distal directions, respectively. The term “approximately,” or like terms (e.g., “substantially”), includes values +/- 10% of a stated value.

Aspects of this disclosure provide configurations of articulation components within a distal tip of a medical device, for example, to facilitate treatment of a subject and/or optimize spacing within the distal tip. The distal tip of the medical device, such as a cholangioscope, may be inserted into a body lumen of a subject (e.g., into a duct or lumen of a patient) in order to perform a medical procedure (e.g., an endoscopic retrograde cholangiopancreatography procedure). The distal tip may include a distal portion of one or more articulation members, for example, the articulation member(s) including one or more pull wires. The articulation member(s) may be configured to articulate the distal tip in one or more directions (e.g., moving the distal tip left, right, up, and/or down). The articulation member(s) may be one or more wires or cables configured to transmit tensile forces to articulate the distal tip.

The distal portion, including the distal tip, of the medical device may further include one or more assemblies, each including various electronic components, such as illumination or lighting elements (e.g., light emitting diodes (LEDs), optical fibers (e.g., plastic optical fibers (POFs), other light guides, or a combination thereof), imaging elements (e.g., cameras, other components having imagers, and/or other optical elements such as lenses), and/or other electronic components (e.g., capacitors, diodes, resistors, etc.). Electronic components of the assemblies described herein may include electrical connections, and may also include various elements mounted, for example, or otherwise connected to, the electrical connections.

It may be desirable for the articulation member(s) to occupy a relatively small, or limited, amount of space, while also ensuring ease of manufacturing and quality of performance. For example, with the articulation member(s) occupying a relatively small amount of space, the medical device may be capable of accommodating additional or larger components (e.g., working channels, lumens, electronic components, and/or other components), and/or have additional space within a cross-section of the distal tip. Additionally or alternatively, with the articulation member(s) occupying a relatively small amount of space within the distal tip, the medical device (e.g., a distal portion of the medical device) may include a smaller overall size, which may help to facilitate insertion and/or navigation within the body. For example, the diameter or other cross-sectional size of the medical device may be reduced and/or an overall diameter or other cross-sectional size of the distal tip may be reduced.

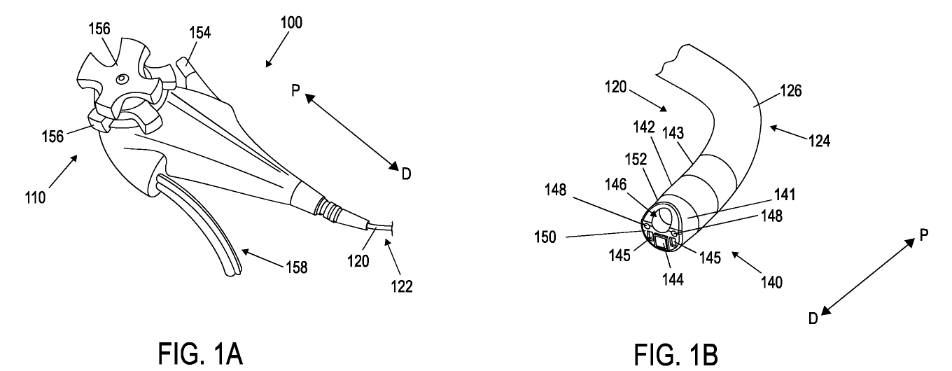

FIGS. 1A and 1B depict proximal and distal portions, respectively, of a medical device 100. In some examples, medical device 100 may be a cholangioscope. Although cholangioscopes are referenced herein, it will be appreciated that medical device 100 may be another type of daughter scope, an endoscope, a ureteroscope, a duodenoscope, a gastroscope, an endoscopic ultrasonography (“EUS”) scope, a colonoscope, a bronchoscope, a laparoscope, an arthroscope, a cystoscope, an aspiration scope, a sheath, a catheter, a tome, or any type of accessory instrument having a shaft and a handle.

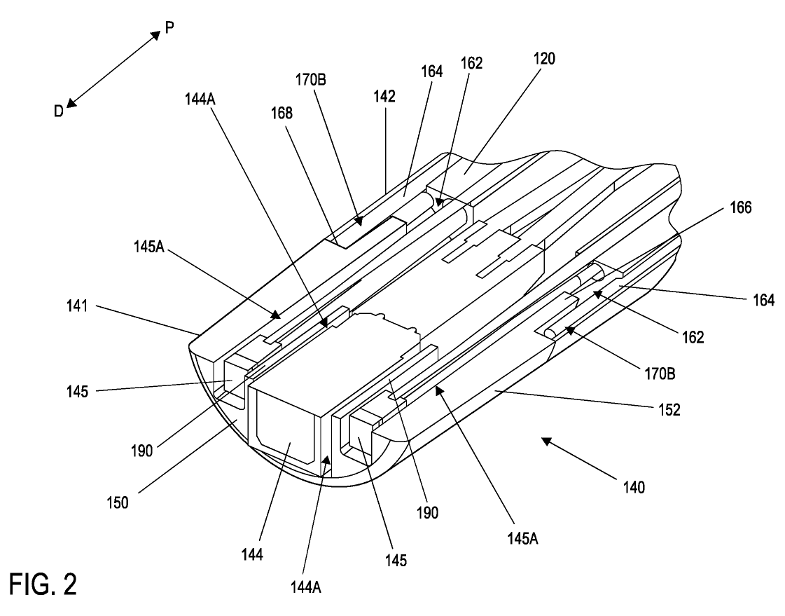

Referring to FIG. 1A, medical device 100 may include a handle 110 and a shaft 120. Referring to FIG. 1B, a distal tip 140 may be coupled to a distal end of shaft 120. Shaft 120 may include a proximal portion 122 (FIG. 1A) and a distal portion 124 (FIG. 1B). Shaft 120 may be a tubular member having one or more lumens extending therethrough. For example, fluids may be delivered and/or removed from the subject via a working channel or any other lumen of shaft 120. Distal portion 124 may include a steerable section 126, which may include an articulation joint or other structure (e.g., a multi-lumen extruded shaft or other bendable shaft) that is configured to deflect or otherwise articulate in order to steer steerable section 126 and distal tip 140. One or more articulation members 164 (FIG. 2) may extend through one or more lumens of shaft 120. As discussed in further detail below, articulation members 164 may be configured to bend, or articulate, steerable section 126 in one or more directions (e.g., left, right, up, and/or down).

Referring to FIG. 1B, distal tip 140 may include a distal cap 141. Distal cap 141 may be secured to a distal-most end of shaft 120 via a steering ring 142. In some aspects, distal tip 140 may further include a spacer 143 adjacent to and/or proximal of steering ring 142. Described in further detail below, spacer 143 may be configured to help bond steering ring 142 to shaft 120. In some aspects, spacer 143 may be configured to help improve the alignment between distal cap 141 and/or steering ring 142 with shaft 120.

Distal cap 141 may include one or more lighting elements 145 (e.g., two lighting elements 145), one or more imaging devices 144, and a distal opening 146 of a lumen (e.g., the working channel) of medical device 100. Distal cap 141 may further include one or more additional distal openings 148. For example, a single distal opening 146 and two additional distal openings 148 are illustrated in FIG. 1B. However, distal cap 141 may include fewer or more distal openings 146, 148. Distal openings 146, 148 may be openings configured for the delivery and/or removal of fluids (e.g., air, water, saline, etc.), objects, and/or accessory devices (e.g., guidewires, baskets, staplers, stents, clips, knives, cautery devices, etc.). One or more lighting elements 145, one or more imaging devices 144, distal opening 146, and distal opening(s) 148 may have any suitable arrangement, for example, on a distal-most face 150 of distal cap 141 and/or on a side surface 152 of distal cap 141. Additionally or alternatively, distal cap 141 may include one or more end effectors (e.g., graspers, snares, staplers, clips, stents, forceps, suturing devices, baskets, etc.) for performing a medical procedure at a treatment site.

Referring back to FIG. 1A, handle 110 may include a port 154, which may provide access to the working channel of medical device 100. An accessory instrument may be inserted into port 154, advanced through the working channel, and out of distal opening 146 of distal cap 141. Handle 110 may also include one or more actuators 156 for controlling aspects of shaft 120 and/or distal tip 140. For example, one or more actuators 156 may include one or more steering knobs (or levers, sliders, etc.) for controlling articulation of steerable section 126. One or more cables and/or wires may extend from one or more actuators 156 to steerable section 126 or to other portion(s) of shaft 120. Tensioning or de-tensioning of the cables and/or wires may cause steerable section 126 to deflect, or articulate, in one or more directions (e.g., left, right, up, and/or down). In alternatives, actuator(s) 156 may be used to control an end effector of distal tip 140 using one or more control wires or cables. Actuator(s) 156 may additionally or alternatively control electronic elements of distal tip 140 and/or deliver air, suction, and/or water.

An umbilicus 158 may extend from handle 110. Umbilicus 158 may include one or more cables, tubes, or other conduits for conveying electrical signals, fluids, and/or suction to or from handle 110, shaft 120, or distal tip 140. Umbilicus 158 may include one or more connectors (not shown) for connecting to capital equipment, which may supply electrical controls, air, water, and/or suction.

Shaft 120 of medical device 100 may be configured for insertion directly into a subject (e.g., via a natural orifice and/or incision). Additionally or alternatively, medical device 100 may be a first medical device, and shaft 120 may be configured for insertion through a lumen or working channel of a second medical device, such as a duodenoscope or any other medical device having a working channel extending from a proximal end to a distal end and configured to receive a device, such as a ureteroscope, an endoscope, a gastroscope, an endoscopic ultrasonography (“EUS”) scope, a colonoscope, a bronchoscope, a laparoscope, an arthroscope, a cystoscope, an aspiration scope, a sheath, or a catheter.

FIG. 2 illustrates a first cross-sectional view (e.g., a longitudinal cross-sectional view) of a distal portion of medical device 100, described above. In particular, FIG. 2 illustrates an exemplary configuration of internal components extending through or into distal cap 141 of distal tip 140 (e.g., from shaft 120 of FIGS. 1A and 1B). For example, as previously described, distal tip 140 may include one or more lighting elements 145 and/or one or more imaging devices 144. Distal-most face 150 of distal cap 141 may define a distal-most end of distal tip 140. Distal-most face 150 may be perpendicular to a central longitudinal axis of distal tip 140. Additionally or alternatively, distal-most face 150 may be angled relative to the central longitudinal axis of distal tip 140.

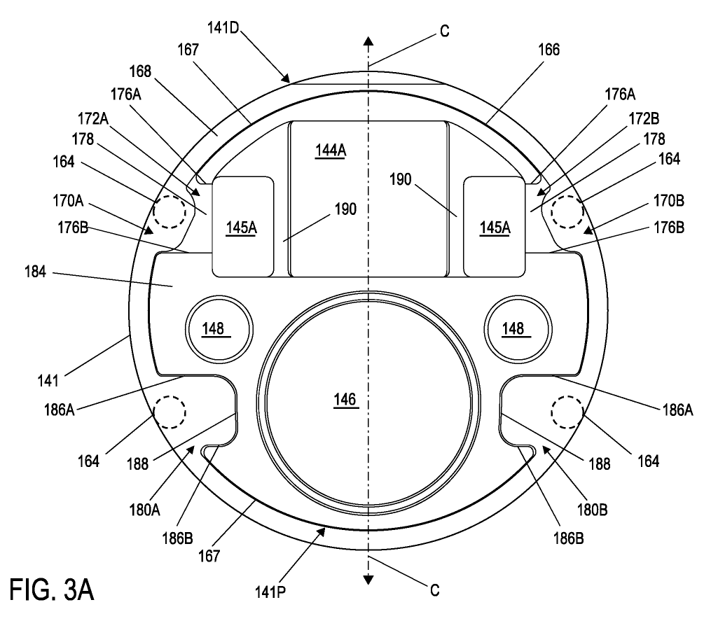

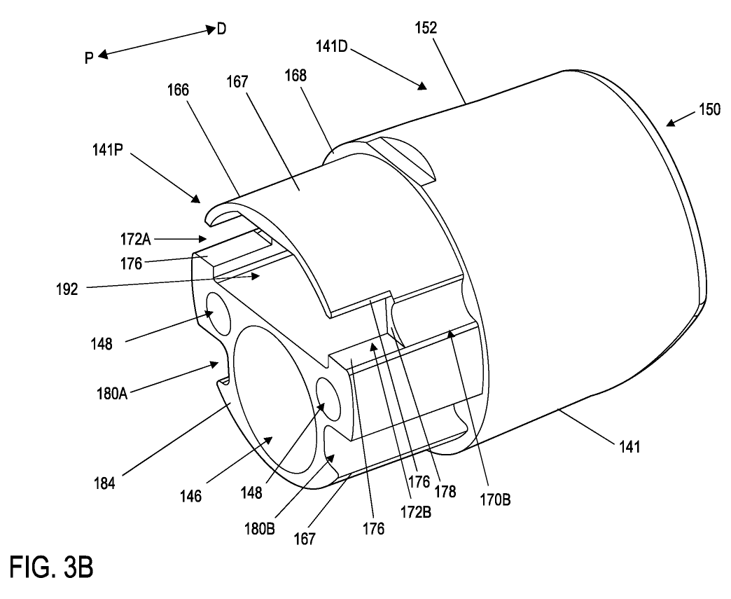

FIGS. 3A and 3B illustrate views of distal cap 141, for example, separate from shaft 120 and other components of medical device 100. FIG. 3A illustrates a proximal-to-distal view of distal cap 141 with the internal components shown in FIG. 2 removed, and FIG. 3B illustrates a perspective view of distal cap 141 with the internal components shown in FIG. 2 removed. Particularly when describing and referring to aspects of distal cap 141, all of FIGS. 2, 3A, and 3B, may be referred to in the following description.

Distal cap 141 may include a proximal portion 141P and a distal portion 141D. As previously discussed, distal cap 141 may be secured to the distal-most end of shaft 120, for example, via steering ring 142 and, in some aspects, spacer 143. Steering ring 142 may circumferentially surround proximal portion 141P of distal cap 141 and the distal-most end of spacer 143. In some configurations, spacer 143 may be removed such that steering ring 142 is secured directly to the distal-most end of shaft 120.

Steering ring 142 may be secured to proximal portion 141P of distal cap 141 and/or the distal-most portion of shaft 120 or spacer 143 via an adhesive, a press-fit, an interference fit, a weld, solder, or using any other technique commonly used in the art to secure two components together. In some aspects, an internal space 162 may be defined between a distal-most end of shaft 120 and proximal portion 141P of distal cap 141, for example, within steering ring 142. Internal space 162 may be filled with or otherwise receive one or more epoxies, one or more adhesive materials, or one or more other filler materials. The epoxy or adhesive material may be used to waterproof or otherwise seal the internal components, described below. In some aspects, internal space 162 may be filled with the epoxy or adhesive material so as to secure internal components within internal space 162 and, thus, within distal cap 141. Internal components (e.g., imaging device(s) 144, illumination element(s) 145, articulation member(s) 164, etc.) and associated cabling may extend from shaft 120, through internal space 162, and into distal cap 141 (e.g., between shaft 120 and proximal portion 141P of distal cap 141).

In other aspects, internal space 162 may be omitted. For example, a distal-most end of shaft 120 may abut a proximal face 184 of distal cap 141. The epoxy, adhesive, or filler material may be applied to proximal face 184 and/or distal-most end of shaft 120 to assist with coupling distal cap 141 to shaft 120.

Steering ring 142 may be secured to proximal portion 141P of distal cap 141. Proximal portion 141P may include a proximal extension 166. For example, a distal end of steering ring 142 may at least partially overlap with proximal extension 166 of distal cap 141. In aspects, a distal-most end of steering ring 142 may abut a proximal-facing ledge 168 of distal cap 141. Shown more clearly in FIG. 3B, proximal-facing ledge 168 of distal cap 141 may be defined around an entire circumference of distal cap 141. In other aspects, proximal-facing ledge 168 of distal cap 141 may extend around only one or more portions of the circumference of distal cap 141. For example, distal cap 141 may include a keying feature that is received by steering ring 142. Described in further detail below, a distal end of one or more articulation members 164 may be secured to an internal surface of steering ring 142.

Shown more clearly in FIGS. 3A and 3B, proximal extension 166 of distal cap 141 may define or include a plurality of features configured to help optimize space within or otherwise reduce the size of distal tip 140. For example, proximal extension 166 may define a first groove 170A and a second groove 170B. (First groove 170A is not visible in FIG. 3B due to the orientation of distal cap 141.) Each of first groove 170A and second groove 170B may be defined on an outer surface of proximal extension 166. In other words, first groove 170A and second groove 170B may extend radially inward relative to an outer surface 167 of proximal extension 166. First groove 170A and second groove 170B may be mirror images of one another other relative to a central plane "C" (FIG. 3A). In aspects, proximal-facing ledge 168 may define a distal end of each of first groove 170A and second groove 170B. Each of first groove 170A and second groove 170B may be configured to receive the distal end of a respective articulation member 164 (FIGS. 2 and 3A).

Proximal extension 166 may further include a first cutout 172A and a second cutout 172B that are proximal of first groove 170A and second groove 170B, respectively. Cutouts 172A, 172B may extend radially inward, for example, from outer surface 167 of proximal extension 166 through an inward-facing surface of proximal extension 166. Cutouts 172A, 172B may extend from a proximal end of each of first groove 170A and second groove 170B to a proximal-most end of proximal extension 166. In other words, cutouts 172A, 172B may be proximal of grooves 170A, 170B, respectively. When distal cap 141 is observed from a side, cutouts 172A, 172B may be U-shaped. For example, each of cutouts 172A, 172B may be defined by two parallel surfaces 176A, 176B that are parallel to one another and to a longitudinal axis of distal cap 141. A distal surface 178 that is perpendicular to each of surfaces 176A, 176B may define a distal end of cutouts 172A, 172B. In aspects, distal surface 178 may define a proximal end of grooves 170A, 170B.

In some aspects, cutouts 172A, 172B may be aligned with respective openings of lumens of shaft 120, and each of first groove 170A and second groove 170B may be offset from lumens of shaft 120. Cutouts 172A, 172B may enable respective articulation members 164 to extend distally from shaft 120 and curve inwards, for example, towards the central longitudinal axis of distal cap 141. In aspects, cutouts 172A, 172B may enable articulation member 164 to extend into/from a respective lumen of shaft 120 and to first groove 170A and second groove 170B, for example, without imparting a sharp angle or bend in articulation member 164.

Proximal extension 166 may further define or include a third groove 180A and a fourth groove 180B each configured to receive the distal end of other articulation members 164 (FIGS. 2 and 3A). Each of third groove 180A and fourth groove 180B may be defined on outer surface 167 of proximal extension 166. In other words, third groove 180A and fourth groove 180B may extend radially inward relative to outer surface 182 of proximal extension 166. For example, third groove 180A and fourth groove 180B may be closer to the central longitudinal axis of distal tip 140 as compared to first groove 170A and second groove 170B. Third groove 180A and fourth groove 180B may be mirror images of each other relative to a central plane “C” (FIG. 3A). In aspects, proximal-facing ledge 168 may define a distal end of each of third groove 180A and fourth groove 180B. A proximal face 184 of proximal extension 166 may define a proximal end of each of third groove 180A and fourth groove 180B. Third groove 180A and fourth groove 180B may each be further defined by two parallel surfaces 186A, 186B and a perpendicular surface 188.

A longitudinal length of each of third groove 180A and fourth groove 180B may be greater as compared to a longitudinal length of each of first groove 170A and second groove 170B. For example, third groove 180A and fourth groove 180B may extend from proximal-facing ledge 168 to a proximal-most end of proximal extension 166 (e.g., to proximal face 184 of proximal extension 166). Third groove 180A and fourth groove 180B may be disposed more radially inward as compared to first groove 170A and second groove 170B. In other words, a largest radial distance measured from an outer circumference of proximal extension 166 to an innermost surface of third groove 180A or fourth groove 180B may be greater than a largest radial distance measured from the outer circumference of proximal extension 166 to an innermost surface of first groove 170A or second groove 170B.

Third groove 180A and fourth groove 180B may be aligned with respective lumens of shaft 120. Accordingly, the distal end of each articulation member 164 may smoothly transition from respective lumens of shaft 120 and into third groove 180A or fourth groove 180B. In some aspects, a size of each of third groove 180A and fourth groove 180B may be larger as compared to first groove 170A and second groove 170B. The larger size of third groove 180A and second groove 180B may be larger because first opening 144A and second opening(s) 145A are not disposed between third groove 180A and fourth groove 180B. For example, first groove 170A and second groove 170B may be smaller to accommodate imaging device(s) 144 and/or lighting element(s) 145.

Each of first groove 170A, second groove 170B, third groove 180A, and fourth groove 180B may enable the distal ends of articulation members 164 to be coupled to the inner surface of steering ring 142 (shown in FIGS. 1A, 2, and 5). For example, each articulation member 164 may bend or extend radially outward towards the inner surface of steering ring 142. The distal ends of the articulation members 164 may be welded, soldered, or otherwise secured to the inner surface steering ring 142. With articulation members 164 secured to the inner surface of steering ring 142, by pulling or otherwise urging one or more articulation members wires proximally, shaft 120 and distal tip 140 may be steerable.

Referring primarily to FIGS. 3A and 3B, distal openings 146, 148 may extend from a proximal end to a distal end of distal cap 141. Accordingly, distal openings 146, 148 may be through-holes extending longitudinally (e.g., parallel to the central longitudinal axis of cap 141) through distal cap 141. Each of distal openings 146, 148 may be aligned with a respective lumen of shaft 120.

Each of imaging device(s) 144 may extend through a first opening 144A of distal cap 141. First opening 144A, and therefore imaging device(s) 144, may be centered between two opposing sides of distal cap 141 (e.g., between a left side and a right side of distal cap 141). A single lighting element 145 may extend through a respective second opening 145A of distal cap 141. In other configurations, multiple lighting element(s) 145 (e.g., two or more) may extend through a single second opening 145A. In some aspects, first opening 144A may be disposed between two second openings 145A. In other aspects, second opening(s) 145A may be disposed only on one side of first opening 144A. In these aspects, lighting element(s) 145 may be disposed only on a single side of imaging device(s) 144.

Shown in FIGS. 2 and 3A, first opening 144A may be at least partially defined by one or more walls 190 extending proximally from distal-most face 150. Wall(s) 190 may be disposed between first opening 144A and each of second opening(s) 145A. A longitudinal length of wall(s) 190 may be less than a longitudinal length of distal cap 141. For example, a proximal-most end of wall(s) 190 may terminate distally of a proximal-most end of distal cap 141. Wall(s) 190 may be configured to divide, or at least partially separate, lighting element(s) 145 from imaging device(s) 144, for example, within a distal portion of distal cap 141. Proximal of wall(s) 190, openings 145A, 144A may merge into a single, larger opening 192 (FIG. 3B). Within larger opening 192, cabling associated with lighting element(s) 145 may converge and/or extend alongside cabling associated with imaging device(s) 144 (e.g., proximal of walls(s) 190). The cabling may then extend through one or more lumens within shaft 120, for example, to connect to handle 110 and/or umbilicus 158.

In some aspects, wall(s) 190 may assist with placement and/or support of lighting element(s) 145 and imaging device(s) 144, for example, during assembly. For example, wall(s) 190 may assist with consistently placing lighting element(s) 145 and imaging device(s) 144 within distal cap 141 (e.g., during assembly or manufacturing of distal tip 140).

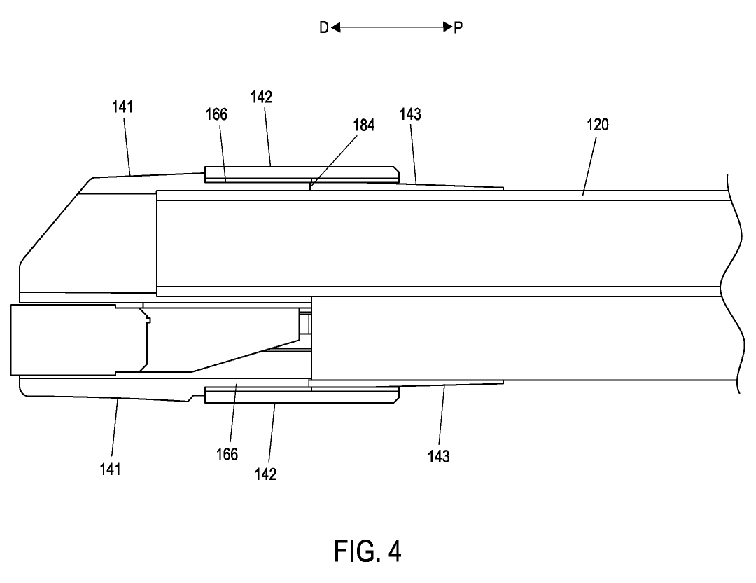

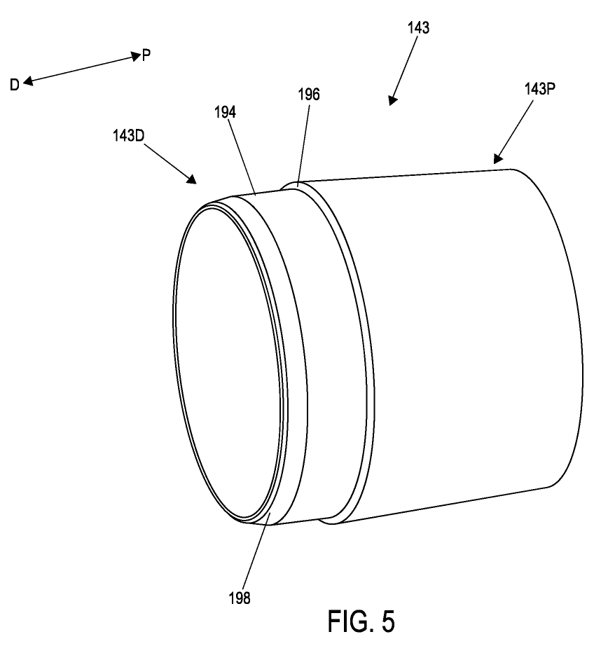

FIG. 4 illustrates another cross-sectional view of distal tip 140, and FIG. 5 illustrates a perspective view of an exemplary spacer 143. Particularly when describing and referring to aspects of spacer 143, FIGS. 4 and 5 may be referred to in the following description. For example, FIG. 4 illustrates the interaction of components of distal tip 140 with one another, including spacer 143. As previously described, distal tip 140 may include distal cap 141 and steering ring 142. In some aspects, distal tip 140 may further include spacer 143. Steering ring 142 may at least partially overlap with proximal extension 166 of distal cap 141. For example, steering ring 142 may be surround and/or be coupled to outer surface 167 of proximal extension 166.

Spacer 143 may be proximal of distal cap 141, when assembled as part of medical device 100. In particular, a distal-most end of spacer 143 may abut a proximal-most end of distal cap 141 (e.g., proximal face 184 of proximal extension 166). Steering ring 142 may at least partially overlap with a distal end portion of spacer 143. In some aspects, steering ring 142 may at least partially circumferentially surround and be secured to an outer surface 194 (FIG. 5) of spacer 143. Spacer 143 may at least partially circumferentially surround and be secured to an outer surface of shaft 120. In some aspects, a proximal portion 143P of spacer 143 may taper proximally such that a thickness of spacer 143 is smaller at a proximal end as compared to an intermediate portion of spacer 143. The taper may assist in creating a smooth transition between steering ring 142 and shaft 120.

Referring primarily to FIG. 5, spacer 143 may be generally cylindrical with a central opening or through-hole. Spacer 143 may include proximal portion 143P and a distal portion 143D. A diameter of distal portion 143D may be smaller than a diameter of an intermediate portion of spacer 143 (e.g., the portion of proximal portion 143P that is distal to the taper of proximal portion 143P). Distal portion 143D may have a smaller diameter, for example, to help enable a smooth transition between steering ring 142 and spacer 143.

In some aspects, steering ring 142 may overlap with and/or at least partially circumferentially surround distal portion 143D. For example, a proximal end of steering ring 142 may abut a distal-facing ledge 196 of spacer 143. Distal-facing ledge 196 may be defined between distal portion 143D and proximal portion 143P. In other words, distal-facing ledge 196 may provide a division point between distal portion 143D and proximal portion 143P.

Distal portion 143D may taper distally (e.g., such that a thickness of a distal end of distal portion 143D is less than a thickness of a proximal end of distal portion 143D). A distal end of distal portion 143D may include an additional taper 198. Additional taper 198 may be steeper as compared to the taper of distal portion 143D and/or proximal portion 143P. Additional taper 198 may assist with positioning and/or guiding steering ring 142 (FIG. 4) over distal portion 143D of spacer 143.

Each of distal cap 141, steering ring 142, and spacer 143 may be comprised of one or more biocompatible materials. For example, distal cap 141, steering ring 142, and/or spacer 143 may be comprised of a metal or metallic material (e.g., stainless steel, aluminum, nitinol, copper, etc.) and/or of a plastic or molded material (e.g., polycarbonate, acrylic, a polymeric material, silicone, epoxy resin, etc.).

While principles of this disclosure are described herein with the reference to illustrative examples in a particular context and for particular medical procedures, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

1. A medical device, comprising:
a shaft defining at least one lumen;
an articulation member extending through the at least one lumen; and
a distal tip coupled to a distal end of the shaft, the distal tip comprising:
 a distal cap having a distal portion and a proximal portion, wherein the proximal portion includes a proximal extension; and
a steering ring at least partially surrounding the proximal extension of the distal cap;
wherein the proximal extension of the distal cap defines a plurality of grooves each configured to receive a distal end of the articulation member, wherein the plurality of grooves is defined along a radially outer surface of the proximal extension.

2. The medical device of claim 1 wherein the plurality of grooves includes a first groove and a second groove, wherein the distal tip further includes a first cutout and a second cutout, wherein the first cutout is proximal of the first groove, wherein the second cutout is proximal of the second groove, wherein each of the first cutout and the second cutout extends radially inward from an outer surface of the proximal extension through an inward-facing surface of the proximal extension.

3. The medical device of claim 2, wherein the articulation member extends radially inward and through the first cutout and the second cutout.

4. The medical device of claim 2, wherein each of the first cutout and the second cutout are defined by two parallel surfaces and a distal surface.

5. The medical device of claim 2, wherein the first cutout and the second cutout are U-shaped when viewed from a side of the distal cap.

6. The medical device of claim 2, wherein the plurality of grooves further includes a third groove and a fourth groove, wherein the third groove and the fourth groove are mirror images of one another relative to a central plane of the distal cap.

7. The medical device of claim 6, wherein a distal end of each of the first groove, the second groove, the third groove, and the fourth groove is defined by a proximal-facing wall.

8. The medical device of claim 6, wherein a longitudinal length of each of the third groove and the fourth groove is greater than a longitudinal length of each of the first groove and the second groove.

9. The medical device of claim 6, wherein each of the third groove and the fourth groove is closer to a central longitudinal axis of the distal tip than the first groove and the second groove.

10. The medical device of claim 6, wherein each of the first groove and the second groove are offset from respective lumens of the shaft, and wherein the third groove and the fourth groove are aligned with respective lumens of the shaft.

11. The medical device of claim 1 further comprising a spacer disposed proximal of the distal cap, wherein the spacer at least partially surrounds a distal portion of the shaft, and wherein the steering ring at least partially surrounds a distal end portion of the spacer.

12. The medical device of claim 11, wherein a proximal portion of the spacer tapers radially inward proximally.

13. The medical device of claim 1 wherein the distal cap further defines a first opening and at least one second opening, wherein the first opening is configured to receive an imaging device, wherein the at least one second opening is configured to receive an illumination device, and wherein the first opening is separated from the second opening by a wall.

14. The medical device of claim 13, wherein, proximal of the wall, the first opening and the second opening converge.

15. The medical device of claim 1 wherein the distal end of the articulation member is coupled to an internal surface of the steering ring.

16. A distal end of a medical device, comprising:
 a distal cap having a distal portion and a proximal portion, wherein the proximal portion includes a proximal extension, wherein the proximal extension of the distal cap defines:
a groove defined along a radially outer surface of the proximal extension, wherein the groove is configured to receive a distal end of an articulation member; and
a cutout proximal of the groove, wherein a portion of the articulation member extends radially inward and through the cutout; and
a steering ring at least partially surrounding the proximal extension of the distal cap.

17. The distal end of claim 16, wherein the steering ring is configured to couple the distal cap to a distal end of a shaft of the medical device, wherein the groove is a first groove and the cutout is a first cutout, wherein the distal cap further includes a second groove, a third groove, and a fourth groove, wherein the first groove and the second groove are mirror images of one another relative to a central plane of the distal cap, and wherein the third groove and the fourth groove are mirror images of one another relative to a central plane of the distal cap.

18. The distal end of claim 16, further comprising a spacer disposed proximal of the distal cap, wherein the spacer is configured to at least partially surround a distal portion of a shaft, and wherein the steering ring at least partially surrounds a distal end portion of the spacer.

19. A distal cap of a medical device, comprising:
a distal portion and a proximal portion, wherein the proximal portion includes a proximal extension, wherein the proximal extension of the distal cap defines:
a groove defined along a radially outer surface of the proximal extension, wherein the groove is configured to receive a distal end of an articulation member; and
a cutout proximal of the groove, wherein a portion of the articulation member is configured to extend radially inward and through the cutout; and
a steering ring at least partially surrounding the proximal extension of the distal cap, wherein the steering ring is configured to couple the distal cap to a shaft of the medical device.

20. The distal cap of claim 19, further comprising a spacer disposed proximal of the distal cap, wherein the spacer is configured to at least partially surround a distal portion of a shaft, and wherein the steering ring at least partially surrounds a distal end portion of the spacer.
